(19) United States
(12) Patent Application Publication
(10) Pub. No.: US 2007/0293748 A1
(43) Pub. Date: Dec. 20, 2007

(54) Means and Method for Detection of Blood Leakage from Wounds

(75) Inventors: Daniel Engvall, Halmstad (SE); Lennart Jonsson, Halmstad (SE); Per-Olof Karlsson, Halmstad (SE); Lars Rutgersson, Gullbrnadstorp (SE); Fredrik Svensson, Halmstad (SE)

Correspondence Address: DICKSTEIN SHAPIRO LLP, 1177 AVENUE OF THE AMERICAS (6TH AVENUE), NEW YORK, NY, 10036-2714, US

(73) Assignee: Redsense Medical AB

(21) Appl. No.: 11/571,152

(22) PCT Filed: Jun. 21, 2005
(86) PCT No.: PCT/SE05/00960

§ 371 Date: May 23, 2007

(30) Foreign Application Priority Data
Jun. 24, 2004 (SE) 0401632-5

(51) Int. Cl. A61B 5/026

(52) U.S. Cl. 600/371

(57) ABSTRACT

An optical fibre through which light passes is used for detecting blood leakage from a wound. The fibre may be comprised by a blood leakage detection apparatus having a light source and a photo detector in radiative communication with the respective ends of the fibre and producing an electrical signal in response to the light detected, photo detector signal amplifying means, means for detecting changes in the amplified signal over time, and alarm means, wherein a decrease in signal strength during a selected time period activates the alarm means; a corresponding method of detecting blood leakage; a device for use with the apparatus of the invention comprising the optical fibre thereof provided with coupling means at its ends and a patch of blood absorbing material.

### FIELD OF THE INVENTION

The present invention relates to a method and means for detecting blood leakage from wounds, and to a corresponding use of the means.

### BACKGROUND OF THE INVENTION

A well-recognised problem in hospital care is that wounds caused by surgery or accidents, in spite of having been properly closed and dressed, may start to bleed again. Due to the dressing by which the wound is covered or due to the patient being unconscious or otherwise unable to recognise the bleeding, it is only noticed by the staff and taken care of after a while. In the meantime the patient may have lost a substantial volume of blood. This will no doubt have a detrimental effect on his or her recovery.

Another problem of similar kind is quite frequently seen in blood dialysis. In a life saving treatment patients with impaired or non-existing renal function purify their blood from salts, urea and other metabolic degradation products on a regular basis, such as two or three times per week. In blood dialysis an artery is punctured by a cannula or needle to make a portion of the patient's blood pass through a dialysis apparatus in which it is purified. The purified blood is returned to the patient by venous infusion through a cannula inserted into a large vein. Most often arterio-venous fistula (or a corresponding graft) is created at a patients wrist or upper arm, from which blood is removed by an arterial cannula and returned downstream by a venous cannula.

A cannula of this kind usually comes with wings extending from a short cylindrical plastic tube in which the cannula is mounted. These wings can be used for securing the cannula by adhesive tape to prevent it from longitudinal displacement in the vein, fistula, or graft. The adhesive tape may accidentally come off and the cannula withdrawn. Inevitably this results in immediate bleeding, which may be quite severe. If the bleeding is not noticed and stopped at once the patient may loose a large volume of blood. Since dialysis patients are usually anaemic, they are particularly affected by such a loss. In addition it is important to prevent blood contained in the dialysis apparatus from being lost if a cannula is removed accidentally. To cope with a loosening arterial needle a safety means is included in known dialysis apparatus. The safety means comprises a pressure sensor disposed on the input side of the apparatus. If the sensor detects a sudden drop in pressure during dialysis the flow of blood through the apparatus is immediately stopped and the personnel alarmed. Due to the pressure drop in the venous needle a loosening thereof cannot be monitored easily in a corresponding manner. The problem of monitoring the insertion state of the venous needle remains to be solved.

### OBJECTS OF THE PRESENT INVENTION

One object of the present invention is to provide a method and an apparatus for detecting blood leakage from wounds caused by surgery or accident that have been properly taken care of.

Another object of the present invention is to provide a method and a means for detecting blood leakage from a wound in a large vein upon an accidental removal of a dialysis needle inserted through the wound.

Further objects of the present invention will become obvious from the following summary of the invention, the description of preferred embodiments thereof illustrated in a drawing, and the appended claims.

### SUMMARY OF THE INVENTION

The present invention is based on the insight that blood leakage of the aforementioned kind can be detected by monitoring the passage of light, such as visible or near IR light, through an optical fibre that comprises, at a location intermediate between its first and second ends, a blood detection zone. In this application “fibre” always refers to an optical fibre of glass or, more preferred, polymer material that is capable of conducting light efficiently. Preferably the blood detection zone has the form of a sharp bend, such as a bend of more than 90°, preferably more than 150°, most preferred of about 180°. A sharp bend is preferably a sharp permanent bend that is, one that does substantially retain its form even in absence of coercing force. Optical fibres of polymer material are particularly suitable for being formed into such permanent sharp bends.

In a monitoring state, in which the fibre wall of the blood detection zone is in dry contact with air and/or solid materials, the transport of light in the fibre will not be affected. In an alarm state, in which the fibre wall of the blood detection zone is contacted with blood, the transport of light in the fibre will be notably and even substantially reduced, such as by 10% or 20% or 35% and even up to about 50% or more within a short period of time such as ten seconds and even one second. The reason for this reduction is the change of (inner) reflectance of at a portion of the fibre wall by contact with blood which is a medium of higher refractive index than the fibre material. Specifically the change is from practically total reflection of light conducted by the fibre at the fibre wall in a monitoring state to that of reduced reflection at fibre wall portions that are in contact with blood in an alarm state.

According to an important aspect of the invention the blood detection zone of the fibre is disposed at a flexible patch. The flexible patch is intended for fixation at a wound or a dressing or plaster covering a wound. The wound may be an intentionally caused wound, such as a punctuation of a vein, or one caused accidentally. The means for fixation may be comprised by the patch or be separate. The patch comprises a flexible backing, optionally resilient, of a kind known in the art for surgical patches. Preferably the patch comprises a liquid absorbent capable of soaking blood. Preferably the liquid absorbent material is disposed in contact with the blood detection zone. The absorbent material, which should have good wetting properties, may be of a woven or non-woven kind or a combination thereof. Cotton gauze and cellulose based non-woven materials are suitable absorbents. Preferably the absorbent is disposed on and attached to the underside of the backing, that is, the side that faces the skin in a mounted state of the patch. The patch of the invention may have any suitable form. “Suitable form” relates the body site or dressing site at which the patch is intended to be fixed. Most often the patch will be of about rectangular form. For use with an infusion cannula the patch of the invention preferably comprises an incision extending from its circumference in the direction of its central portion. The inner end of the incision is intended to be disposed adjacent to the cannula.

According to an advantageous aspect of the invention the fibre is firmly fixed at the patch at near its blood detection zone. In this configuration the patch and the fibre form a single disposable device.

According to another advantageous aspect of the invention the patch comprises a short tube of a polymer or other suitable material, preferably a resilient material, fixed to the patch, into which tube the sharp bend of the fibre can be inserted to a depth to make it protrude from the other end of the tube and to be held removably in that position. The open-ended tube is fixed at the patch by for instance, gluing. More particularly, according to the present invention, is disclosed a means of the aforementioned kind, comprising a light source, a light detector, a patch capable of being fixed to a wound or a wound dressing, an optical fibre being attached to the patch and arranged to conduct light from the light source to the detector in a monitoring state, the light received by the detector being reduced in an alarm state by blood leaking from the wound or the dressing.

According to a first preferred aspect of the present invention is disclosed a means of the aforementioned kind, comprising an apparatus and a single-use disposable device, the apparatus comprising electronic means for the emission and detection of light, in particular visible or near-infrared light, the device comprising a patch, a means for temporary fixation of the patch at a wound or a wound dressing, an optical fibre for conducting the light from the emission means via the patch to the detection means, the fibre at its passage via the patch comprising a zone in which the reflectance of the fibre wall in respect of light conducted by the fibre is affected by blood contacting the wall in the zone, a difference in returned light indicating a leakage of blood or serum from the wound or the dressing.

At its free ends the fibre of the invention is mounted in connectors by which it can be releaseably attached, directly or indirectly, to the light source and the detector, respectively, to put the light source in radiative communication with the detector. The connectors may be of any suitable kind such as, for instance, screw connectors or connectors of a bayonet type or of piston/cylinder type comprising resilient securing means.

According to a further preferred aspect of the invention, the light source and the light detector are disposed in a housing, which additionally comprises coupling means for mounting the light source end and the detector end connectors of the optical fibre mounting means, power supply means, in particular re-chargeable or not re-chargeable cell(s), detector signal amplification means and alarm means. The light of the light source may be continuous. To save power it may also be intermittent, in which case the housing also comprises a light modulating means. The housing further comprises signal amplification means and comparator means for comparing the amplified signal to a signal threshold. If the light received by the detector and amplified falls below the threshold an alarm in the housing is activated electronically. The alarm may emit a sound and or optical signal. The alarm may also emit a radio wave signal and comprise, for this purpose, radio signal emitting means for sending an amplified detector signal or an alarm signal to a remote receiver forming part or being coupled to a central patient monitoring unit or similar in a dialysis clinic, an intensive care department, etc.

### DESCRIPTION OF THE FIGURES

The invention will now be explained in more detail by reference to preferred embodiments illustrated in a drawing in which

FIG. 1 is a side view of a first embodiment of the apparatus of the invention mounted on a forearm of a patient comprising a disposable patch and monitoring unit, in part in section;

FIG. 1a is a, enlarged partial section of the disposable patch of FIG. 1

FIG. 2 is a sectional view of the patch of the embodiment of FIGS. 1 and 1a;

FIG. 3 is a rough sectional view of the monitoring unit mounted of the apparatus of FIGS. 1 to 2;

FIG. 4 is an enlarged partial section of the disposable patch of FIGS. 1 and 2, upon the cannula having been accidentally removed;

FIG. 5 is a block scheme of the electronics of the embodiment of FIGS. 1 to 4, except for the radiotransmitter/receiver function.

### DETAILED DESCRIPTION OF A PREFERRED EMBODIMENT

The apparatus of the invention shown in the Figures comprises a single-use disposable flexible patch 1 of about rectangular form. In the Figures it is shown disposed around an infusion cannula 4 inserted through the skin 52 and tissue 53 into a vein 50 in the forearm of a patient. The tip 4′ of the cannula 4 is located in the lumen 51 of vein 50. The patch 1 surrounds the cannula 4 at the punctuation site by means of a wedge-formed incision 3 in the patch 1. The incision 3 defines two opposite wings 7, 9 that extend from the central portion of the patch 1. At its distal end the cannula 1 is mounted in a short polycarbonate tube 14 provided with wings (not shown), which stops further insertion of the cannula 4 into the vein 50. From the polycarbonate tube 14 two wings (not shown) extend radially. A flexible tube 6 by which purified blood is adduced from a hemodialysis apparatus (not shown) is in fluid communication with the cannula 4 lumen; neither shown is the corresponding arterial cannula by which blood is transferred from the patient to the hemodialysis apparatus. The cannula 4 and the flexible tube 6 are secured at the patient's arm by adhesive tape, of which only one piece of tape 5 shown. Another piece of adhesive tape (not shown) that secures the cannula 4 is fastened around the cannula wings. The patch 1 includes a flexible backing of a reinforce polymer material, which is provided along its periphery zone 5 with an adhesive layer for releaseably attaching the patch 1 to the skin 52 of the patient. The central portion 13 of the patch 1 is provided with several layers of cotton gauze. A flexible short tube 8 is arranged in the cotton gauze layers so as to extend from the periphery of the patch cotton gauze to a short distance from the inner end of the incision 3. An optical fibre 10, 11, 12 (Toray Industries, Japan; ø 0.25 mm, poly(methylmethacrylate) core, fluorinated polymer cladding) comprising a bend 10 of about 270° and first and second portions 11, 12 extending from either side of the bend 10 is disposed in the tube 8 so as to make the bend 10 extend from the inner end of the tube 8 facing the incision 3, whereas the first and second fibre portions 11, 12 extend from the other, peripheral end of the tube 8 to a monitoring unit 2 comprising a housing 20 disposed at a distance on the patient's forearm on which it is fastened by a bracelet 30. At their free ends the first and second fibre portions 11, 12 are inserted in central bores of cylindrical male parts 15, 16 of snap connections and fixed there by shrinking of the respective male part 15, 16, the corresponding female parts 33, 34 of which are fixed at openings 31, 34 of the housing 20. The female parts 33, 34 are somewhat resilient to allow the somewhat radially bulging front end portions 17, 18 of the male parts 15, 16 to be inserted in the female parts 33, 34 and to be removeably held there. Thus mounted at the monitoring unit 2 the free ends of the first and second fibre portions 11, 12 face a IR diode 21 emitting near IR radiation and a photo diode 22, respectively. The IR diode 21 and the photo diode 22 are electrically connected to an energising circuit 23 and a signal amplification circuit 24, respectively, of an electronics board 25. The circuits 23, 24 are powered by a dry battery 26, which may be rechargeable. The signal amplification circuit comprises a comparator that is designed to detect a sudden fall of the photo detector 22 signal, and is electrically coupled to an alarm unit 27 comprising a bell alarm which is triggered by said fall of signal. Additionally the signal amplification circuit is coupled to a radio wave transmitter 28 comprising an antenna 29. Via the wireless transmitter 28 the monitoring unit 2 is in intermediate or continuous contact with a central monitoring desk 40 of the dialysis department. The monitoring desk 40 comprises a radio wave receiver 41 and an acoustic alarm 42 but may also include, additionally or alternatively, an optical alarm.

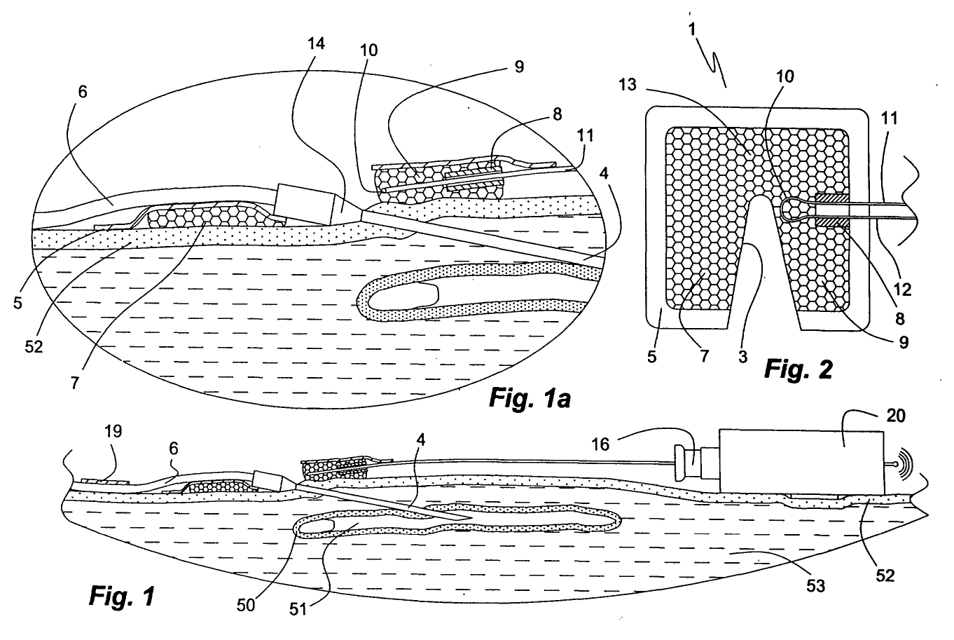

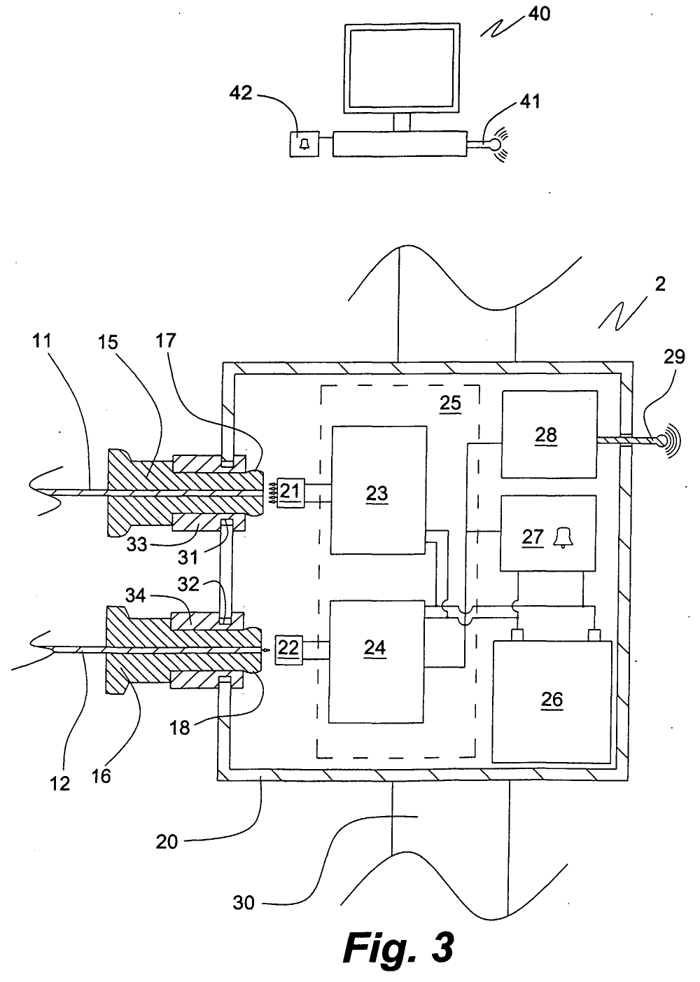

The electronics of the embodiment of FIGS. 1-3 are illustrated in FIG. 5 except for the wireless transmitter/receiver function. All resistances are in ohm. Reference nos. designate:
100, dry cell, 9V;
101, on/off switch;
102, DC-DC transformer (3-terminal positive regulator LM78L05ACM 5V, 0.1A; National Semiconductor, U.S.A.), minimum input 6.7 V; 1=Vₒᵤₜ; 2, 3, 6, 7=ground; 8=Vᵢₙ;
103, IR diode (transmitter HFBR-1412, Agilent Technologies, U.S.A.); 2, 6, 7=anode; 3=cathode;
104, photodiode light detector (receiver HFBR-2412, Agilent Technologies), incorporates a DC amplifier driving an open-collector Schottky output transistor); 2=signal out; 3, 7=ground; 6=Vᵢₙ;
105, amplifier circuit LM324 (ST Microelectronics, U.S.A.), comprises four independent frequency compensated operational amplifiers (OA); 1=OA 4, signal out; 2=OA 4, in (−); 3=OA 4, in (+); 4=supply voltage; 5=OA 3, in (+); 6=OA 3, in (−); 7=OA 3, signal out; 8=OA 2, signal out; 9=OA 2, in (−); 10=OA 2, in (+); 11=ground; 12=OA 1, in (+); 13=OA 1 in, (−); 14=OA 1, signal out. The first OA amplifies the signal by a factor of 1.5; the second OA is comprised by comparator circuit that compares the input signal from OA 1 with a voltage level set by a voltage separator circuit 109 comprising by two pre-set 10 k potentiometers by which either a very low (about 0 V) or a high signal is obtained form OA 2; the signal is further amplified by OA 3 prior to being sent to the signal buzzer. The fourth OA is a comparator for monitoring the circuitry supply voltage from the DC/DC converter that must not decrease below 6.5 V, which is the operation limit for the DC/DC converter 102. The voltage separation by a 47 kΩ/68 kΩ separator circuit 110 is used as a reference. Light emission by diode 107 is triggered by the dry cell voltage falling under 6.7 V;
106, piezoelectric signal buzzer;
107, light-emitting diode, green, 3 mm, EL1224SYGC LED (Everlight, Taiwan); indicates stable operating conditions;
108, light-emitting diode, orange, 3 mm, EL1254USOD/s400 (Everlight, Taiwan); warns for exhausted dry cell.

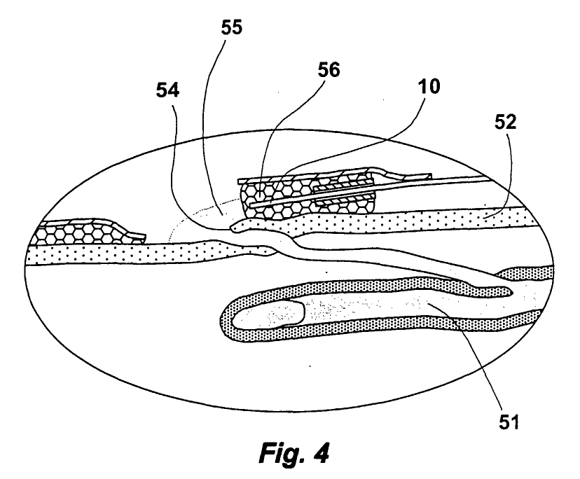

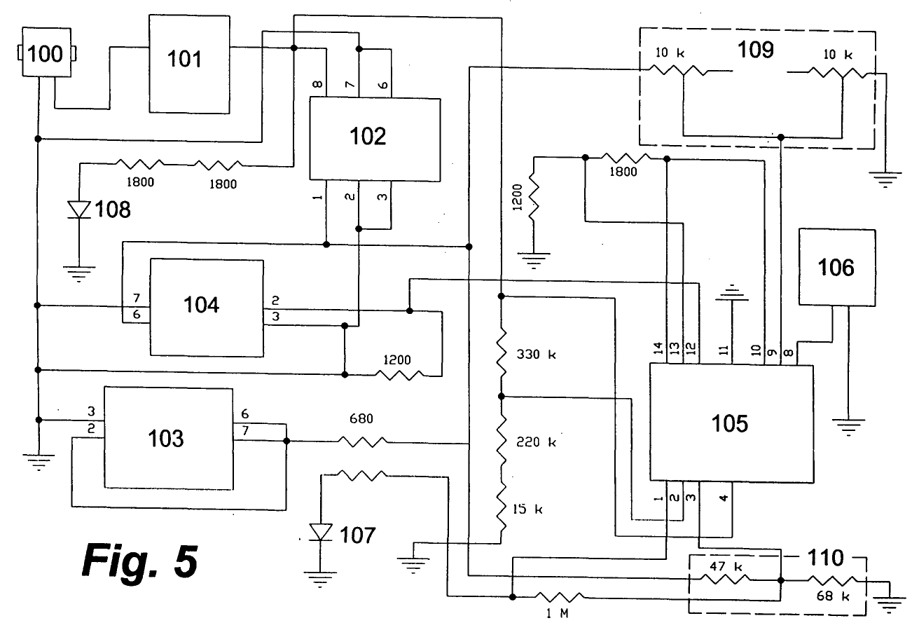

FIG. 4 illustrates the situation immediately after the accidental removal of the venous cannula in FIGS. 1 and 1a. Blood 55 emerges from the insertion wound 54. A part 56 of it is soaked up by the cotton layers in the right (seen from below) flap 9 thereby contacting the bend 10 of the optical fibre. The results drop of reflectance in the bend 10 triggers the alarm 27 and sends a radiowave signal to the monitoring desk 40.

1. of an optical fibre in a state in which visible or infra-red light passes through it, A method for detecting blood leakage from a wound comprising passing visible or infra-re light through an optical fibre and determining if there was attenuation of the light during its passage through the fibre.

2. The method of claim 1, wherein the light passing through the fibre is emitted by a light source in radiative communication with one end of the fibre and detected by a photo detector in radiative communication with the other end of the fibre.

3. The use method of claim 1, wherein the fibre comprises a blood detection zone comprised by a sharp permanent bend of the fibre.

4. The method of claim 1, wherein the fibre is of polymer material.

5. The method of claim 2, wherein the photo detector is a photodiode.

6. The method of claim 2, wherein the light source is a light emitting diode.

7. An apparatus for detecting blood leakage from a wound, comprising an optical fibre, a light source in radiative communication with one end of the fibre and a photo detector in radiative communication with the other end of the fibre and producing an electric signal in response to the light detected, a photo detector signal amplifier, a detector for detecting changes in the amplified signal over time, and an alarm, wherein a decrease in signal strength exceeding a pre-set level activates the alarm to indicate leakage of blood.

8. The apparatus of claim 7, wherein the optical fibre 5 comprises a blood detection portion disposable near a wound the leakage of which shall be monitored.

9. The apparatus of claim 8, wherein the blood detection zone is attached to a patch comprising a blood absorbing material.

10. The apparatus of claim 9, wherein the patch further comprises adhesive for attaching it to the skin of the patient or to a bandage or similar covering a wound.

11. The apparatus of claim 10, wherein the adhesive means is comprised by a flexible backing attached to one side of the patch.

12. The apparatus of claim 7, wherein the light 20 source, the photo detector, the amplifier, the detector changes in the amplified signal and, optionally, the alarm is disposed in a housing designed to be worn by the patient.

13. The apparatus of claim 12, wherein the housing comprises a power supply means selected from rechargeable or non-rechargeable battery.

14. The apparatus of claim 12, wherein the optical fibre comprises, at its free ends, mounts for mounting it to the housing so as to provide for optical communication with the light source and the photo detector through respective openings in the housing.

15. The apparatus of claim 12, wherein the housing comprises a wireless signal transmitter capable of being received by an alarm disposed at a distance from the patient.

16. The apparatus of claim 8, wherein the blood detection zone is comprised by a sharp permanent bend of the fibre.

17. A blood leakage detecting device for use in the apparatus of claim 7, comprising an optical fibre, a coupler for the fibre to a housing containing a light source and a photo detector, the coupler being disposed at the free ends of the fibre.

18. The device of claim 17, further comprising a patch of a blood absorbing material disposed at sharp permanent bend of the fibre.

19. The device of claim 18, further comprising a backing of a thin and flexible material attached to one side of the patch with its one face comprising an adhesive disposed at a circumferential zone thereof surrounding a central zone to which the patch is attached.

20. The device of claim 19, wherein the combination of backing and patch comprises an incision extending from the periphery of the backing towards the centre.

21. A method for monitoring a wound from which blood leakage may occur, comprising, in no particular order;
attaching the device of claim 17 to a bandage or similar covering the wound of a patient or to a skin area surrounding a wound;
attaching the apparatus of claim 12 to the patient at a distance from the wound;
coupling the free ends of optical fibre to the housing to establish radiative communication between the fibre and the light source and the photo detector, respectively;
making the light source emit light to be detected by the photo diode for a desired monitoring period;
recording a sudden decrease in the intensity of light detected by the photo detector as a sign of a blood leakage.

22. The method of claim 21, wherein the wound is one caused by a venous needle and wherein the monitoring period substantially corresponds to a period of infusion of blood or a pharmaceutical.

23. The method of claim 22, wherein the combination of patch and backing comprises a slit extending from the periphery towards the centre and wherein the patch is attached so as to dispose the needle in the slit.

24. The method of claim 21, wherein the sudden decrease in light intensity is 10% or more within ten seconds.

25. The method of claim 21, further comprising triggering an alarm by said sudden decrease in light intensity.
